(19) United States
(12) Patent Application Publication
(10) Pub. No.: US 2023/0372317 A1
(43) Pub. Date: Nov. 23, 2023

(54) A LIQUID INJECTABLE COMPOSITION OF DONEPEZIL

(71) Applicant: PTS Consulting, LLC, Shawnee, KS (US)

(72) Inventors: Paul T. SUDHAKAR, Shawnee, KS (US); Sukhada Satish SHEVADE, Mumbai, Maharashtra (IN); Devarajan Padma VENKITACHALAM, Mumbai, Maharashtra (IN); Maharukh T. RUSTOMJEE, Mumbai, Maharashtra (IN)

(21) Appl. No.: 18/247,943

(22) PCT Filed: Oct. 25, 2021
(86) PCT No.: PCT/US2021/056512

§ 371 Date: Apr. 5, 2023

Related U.S. Application Data
(60) Provisional application No. 63/144,698, filed on Feb. 2, 2021.

(30) Foreign Application Priority Data
Oct. 27, 2020 (IN) 202021046731

(51) Int. Cl. A61K 31/445; A61K 9/00; A61K 47/34

(52) U.S. Cl. CPC A61K 31/445; A61K 9/0024; A61K 47/34

(57) ABSTRACT

A liquid injectable composition comprising:
(a) a bioerodible and biodegradable, release retardant polymer comprising polylactic-ccs-glycolic acid) (PLEA), polylactide (PLA), or combination thereof;
(b) a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or combination thereof; and
(c) donepezil, in the free base or pharmaceutically acceptable salts thereof.

### CROSS REFERENCE TO RELATED APPLICATIONS

This application is a 35 U.S.C. § 371 National Phase Application of PCT/US2021/056512, filed Oct. 25, 2021, which claims the benefit of priority to Indian Patent Application No. IN202021046731, filed Oct. 27, 2020, and U.S. Provisional Patent Application No. 63/144,698, filed Feb. 2, 2021, the disclosures of which are incorporated herein by reference in their entirety.

### FIELD OF THE INVENTION

The present invention relates to a liquid injectable composition of donepezil and the process of its preparation.

### BACKGROUND OF THE INVENTION

Donepezil is an acetylcholinesterase inhibitor most commonly used in the treatment of Alzheimer's disease.

U.S. Pat. No. 6,143,314 (assigned to M/s Atrix laboratories Inc., referred to herein as '314) discloses polymeric compositions of biologically active agents comprising polylactide glycolides and polyethylene glycols as polymeric controlled release additives for forming a controlled release implant within a body. However, '314 does not teach its use in compositions useful in the treatment of Alzheimer's disease nor does it suggest compositions without polyethylene glycols. The composition of the present invention is specific to donepezil and is devoid of polyethylene glycols.

U.S. Pat. No. 7,128,927 (assigned to M/s Atrix laboratories Inc., referred to herein as '927) teaches an emulsion controlled release composition of a biologically active agent. However, the composition of the present invention is an in-situ gel forming solution and not an emulsion.

U.S. Pat. No. 9,353,059 (assigned to M/s Zi-Oiang Gu, referred to herein as '059) discloses compositions of donepezil pamoate. However, the composition of the present invention will comprise donepezil free base or hydrochloride salt.

United States Patent number 20160022583 (assigned to M/s Dongkook Pharmaceutical Co. Ltd, referred to herein as '583) discloses microsphere controlled release composition of donepezil base, donepezil pamoate, donepezil napadisilate and donepezil xinafoate. However, the composition of the present invention does not comprise microspheres or donepezil salts disclosed therein

Korean Patent KR2237737 B1 (assigned to HLB Pharmaceutical referred to herein as '737) discloses microfluidic chip based manufacturing method for preparation of sustained-release PLGA microsphere containing donepezil. However, the composition of the present invention does not comprise microspheres of donepezil.

KR102194794B1 (assigned to Chungbuk National University Industry Academic Cooperation Foundation, Sungkyunkwan University referred to herein as '794) discloses method for manufacturing donepezil sustained-release microparticles based on ion exchange resin. However, the composition of the present invention does not comprise microparticles or ion exchange based microparticles.

WO2020204662A1 (assigned to Chong Kun Dang Pharmaceutical Corp referred to herein as '662) discloses Donepezil myristyloxymethyl ether or pharmaceutically acceptable salt thereof. However, the composition of the present invention does not contain donepezil myristyloxymethyl ether or pharmaceutically acceptable salt.

Soad A. Yehia et al. Journal of Liposome Research, 2012; 22(2): 128-138, discloses an injectable in situ forming poly-DL-lactide and DL-lactide/glycolide implant containing lipospheres to control burst release of donepezil HCl. However, the composition of the present invention does not comprise lipospheres.

### OBJECT OF THE INVENTION

The present invention provides for a liquid injectable composition that includes: (a) a bioerodible and biodegradable, release retardant polymer that includes polylactic-co-glycolic acid) (PLGA), polylactide (PLA), or a combination thereof; (b) a biocompatible solvent that includes N-methyl pyrrolidone; and (c) donepezil, in the free base or a pharmaceutically acceptable salt thereof.

The present invention also provides for a kit that includes: (a) a first container that includes donepezil, in the free base or a pharmaceutically acceptable salt thereof optionally in a biocompatible solvent and (b) a second container that includes a bioerodible and biodegradable, release retardant polymer that includes polylactic-co-glycolic acid) (PLGA), polylactide (PLA), or a combination thereof optionally in a biocompatible solvent.

The present invention also provides for a method of treating a subject suffering from a disease or disorder ameliorated by donepezil. The method includes administering the liquid injectable composition described herein to a subject in an amount and for a period of time sufficient to treat the subject. The disease can be, e.g., Alzheimer's disease (AD), Schizophrenia, Parkinson's Disease, Down Syndrome. The administering can be e.g., intramuscular or subcutaneous. Additionally, the subject can be e.g., a human.

### SUMMARY OF THE INVENTION

A liquid injectable composition comprising:
(a) bioerodible and biodegradable release retardant polymer selected from polylactic-co-glycolic acid) (PLGA), polylactide or combination thereof;
(b) biocompatible solvent selected from dimethyisulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol or combination thereof; and
(c) donepezil, in the free base or pharmaceutically acceptable salts thereof.

Specifically, a liquid injectable composition comprising:
(a) 3-45 wt. % of bioerodible and biodegradable release retardant polymer selected from poly(lactic-co-glycolic acid) (PLGA), polylactide (PLA) or combination thereof;
(b) 25-94 wt. % of biocompatible solvent selected from dimethylsulfoxide, 2-pyrrolidone, N,N-dimethyl acetamide, dimethylformamide, glycofurol, glycerol formal, propylene glycol, benzyl benzoate. N-methyl-2-pyrrolidone, benzyl alcohol or combination thereof;
(c) 3-30 wt. % of donepezil, in the free base or pharmaceutically acceptable salts thereof; and wherein PLGA comprises a molar ratio of 35-85% lactic acid to 65-15% glycolic acid; and the wt % is on the weight of the liquid injectable composition & wherein the composition is a controlled release composition.

More specifically, a liquid injectable composition comprising:
(a) 3-45 wt. % of poly(lactic-co-glycolic acid) (PLGA);
(b) 25-94 wt. % of N-methyl-2-pyrrolidone or Dimethylacetamide or combination thereof;
(c) 3-31) wt. % of donepezil, in the free base or pharmaceutically acceptable salts thereof; and wherein PLGA comprises a molar ratio of 35-85% lactic acid to 65-15% glycolic acid; and the wt % is on the weight of the liquid injectable composition, & wherein the composition is a controlled release composition.

A kit comprising:
(a) a first container comprising donepezil, in the free base or a pharmaceutically acceptable salt thereof optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof, and
(b) a second container comprising a bioerodible and biodegradable, release retardant polymer comprising polylactic-co-glycolic acid) (PLGA), polylactide (PLA), or a combination thereof optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof.

Specifically, a kit comprising:
(a) a first container comprising donepezil, in the free base or a pharmaceutically acceptable salt thereof optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof;
(b) a second container comprising a bioerodible and biodegradable, release retardant polymer comprising polylactic-co-glycolic acid) (PLGA) or its combination thereof; optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof and
wherein PLGA comprises a molar ratio of 35-85% lactic acid to 65-15% glycolic acid; and wherein the composition is a controlled release composition of unit dosage form.

More specifically, a kit comprising:
(a) a first container comprising 3-30 wt. % of donepezil, in the free base or a pharmaceutically acceptable salt thereof; optionally in 25-94 wt. % of N-methyl-2-pyrrolidone or Dimethylacetamide or combination thereof;
(b) a second container comprising 3-45 wt. % polylactic-co-glycolic acid optionally in N-methyl-2-pyrrolidone;
wherein PLGA comprises a molar ratio of 35-85% lactic acid to 65-15% glycolic acid; and
the wt % is on the weight of the liquid injectable composition & wherein the composition is a controlled release composition of unit dosage form.

A method of treating a subject suffering from a disease or disorder ameliorated by donepezil, the method comprising administering the liquid injectable composition of the present invention in an effective amount and for a period of time sufficient to treat the subject.

### DESCRIPTION OF THE INVENTION

The present invention can be more readily understood by reading the following detailed description of the invention and study of the included examples.

The present invention is based, in part, upon the discovery of novel liquid injectable composition that provides advantages when used for the in vivo delivery to a mammal of the active pharmaceutical ingredient (API) donepezil, as the free base or pharmaceutically acceptable salt form. In doing so, the present invention provides for a liquid injectable composition that can conveniently be administered to a subject (e.g., intramuscular or subcutaneous). After administration and contact with bodily fluids, the liquid injectable composition will form a depot, which will erode and degrade within the body (in situ), to release the donepezil. In doing so; the liquid injectable composition provides for a suitable therapeutic index and/or relatively low incidence, severity, or duration of adverse reaction(s), compared to previously described dosage forms containing the active ingredient in an equivalent amount. The release of the donepezil can be, e.g., modified release, controlled release or sustained release.

The liquid injectable composition may be used for a variety of purposes, including for the in vivo delivery of the active pharmaceutical ingredient (API) donepezil. Accordingly, the present invention further provides methods of treating diseases or disorders, such as Alzheimer's.

The present invention relates to liquid injectable compositions of donepezil in free base or its pharmaceutically acceptable salts thereof used in the treatment of Alzheimer's.

According to one embodiment of the present invention is a liquid injectable composition comprising bioerodible and biodegradable release retardant polymer(s); biocompatible solvent(s) and donepezil in the free base or its pharmaceutically acceptable salts thereof.

The liquid injectable composition of the present invention may have volume of up to 3.0 ml; preferably in the range of 0.3 to 3.0 nil.

The liquid injectable composition of the present invention may have mass of up to 3.0 g; preferably in the range of 0.3 to 3.0 g.

The bioerodible and biodegradable release retardant polymer(s) may be selected from polylactic-co-glycolic acid) (PLGA), polylactide (PLA) or combination thereof. The PLGA comprises different molar ratios of lactic acid to glycolic acid such as 35-85% lactic acid to 65-15% glycolic acid.

The bioerodible and biodegradable release retardant polymer(s) may be in the range of 3-45 wt % of the liquid injectable composition.

Donepezil may be in free base form or as pharmaceutically acceptable salt such as hydrochloride.

Donepezil free base or pharmaceutically acceptable salt may be in the range of 3-30 wt % of the liquid injectable composition. The amount of donepezil free base or pharmaceutically acceptable salt may range from 10 mg to 1000 mg.

The biocompatible solvent may be selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone benzyl alcohol or combination thereof.

The biocompatible solvent used may be in the range of 25-94 wt % of the liquid injectable composition.

The liquid injectable composition of the present invention may further comprise at least one of stabilizing agent, crystallization inhibitor, pH adjusting agent, buffer, preservative and burst release control additive.

A liquid injectable composition of the present invention may be selected from (a)-(j):
(a) liquid injectable dosage form, capable of forming a depot in situ;
(b) liquid injectable dosage form, capable of forming a depot in situ, which releases the donepezil over a period of time;
(c) liquid injectable dosage form, capable of forming a gel in situ in contact with aqueous fluids
(d) liquid injectable dosage form, capable of forming a gel in situ, in contact with aqueous fluid and releases the donepezil over a period of time;
(e) time release liquid injectable dosage form
(f) controlled release liquid injectable dosage form;
(g) modified release liquid injectable dosage form;
(h) sustained release liquid injectable dosage form;
(i) extended release liquid injectable dosage form; and
(j) liquid injectable dosage form, capable of forming a depot in situ with a reduced burst effect.

Specifically, a liquid injectable composition comprising:
(a) 3-45 wt % of bioerodible and biodegradable release retardant polymer selected from poly(lactic-co-glycolic acid) (PLGA), polylactide (PLA) or combination thereof;
(b) 25-94 wt. % of biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol or combination thereof;
(c) 3-30 wt. % of donepezil, in the free base or pharmaceutically acceptable salts thereof; and wherein PLGA comprises a molar ratio of 35-85% lactic acid to 65-15% glycolic acid; and
the wt % is on the weight of the liquid injectable composition & wherein the composition is a controlled release composition.

More specifically, a liquid injectable composition comprising:
(a) 3-45 wt. % of poly(lactic-co-glycolic acid);
(b) 25-94 wt. % of N-methyl-2-pyrrolidone irr Dimethylacetamide or combination thereof;
(c) 3-30 wt. % of donepezil, in the free base or pharmaceutically acceptable salts thereof; and
wherein PLGA comprises a molar ratio of 35-85% lactic acid to 65-15% glycolic acid; and the wt % is on the weight of the liquid injectable composition. & wherein the composition is a controlled release composition.

According to another embodiment of the present invention the injectable composition may be presented in the form of kit comprising two containers. The two containers may be ampoules or vials or prefilled syringes. The containers may be of 0.1 ml, 0.25 ml, 0.5 ml, 1.0 ml, 2.0 ml or 5.0 ml.

Typically, when the two containers are prefilled syringes, they are to be coupled together and the components are to be mixed by pushing the contents of both the syringes back and forth between syringes to obtain a completely homogenous solution. A lure-lock device may be used for mixing.

The two containers may also be vials or ampoules, in such a case one solution from ampoule/vial is withdrawn in a syringe and added to the other ampoule/vial where it is mixed and then again withdrawn into another syringe for injection into the patient.

The container may comprise donepezil in the free base or a pharmaceutically acceptable salt thereof is in the form of a dry powder.

The container may comprise dissolved or suspended donepezil in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof.

The kit may be present in unit dosage form; preferably in a unit dosage form, wherein (a) the first container is a prefilled injectable syringe or ampoule or vial; and (b) the second container is a prefilled injectable syringe or ampoule or vial.

The unit dosage form may be a single dose.

The kit may be configured such that the contents of the first container and the contents of the second container can be sufficiently mixed together to form a liquid injectable composition to be administered within 15 minutes after the mixing.

Specifically, a kit comprising:
(a) a first container comprising donepezil, in the free base or a pharmaceutically acceptable salt thereof; optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof;
(b) a second container comprising a bioerodible and biodegradable, release retardant polymer comprising polylactic-co-glycolic acid) (PLGA), polylactide (PLA), or a combination thereof; optionally in a biocompatible solvent selected from dimethylsulfoxide, 2-pyrrolidone, N,N-di methyl acetamide, dimethylformamide, glycofurol, glycerol formal, propylene glycol, benzyl benzoate, N-methyl-2-pyrrolidone, benzyl alcohol, or any combination thereof and wherein PLGA comprises a molar ratio of 35-85% lactic acid to 65-15% glycolic acid; and wherein the composition is a controlled release composition of unit dosage form.

More specifically, a kit comprising:
(a) a first container comprising 3-30 wt. % of donepezil, in the free base or a pharmaceutically acceptable salt thereof; optionally in 25-94 wt. % of N-methyl-2-pyrrolidone or Dimethylacetamide or combination thereof;
(b) a second container comprising 3-45 wt. % poly(lactic-co-glycolic acid optionally in N-methyl-2-pyrrolidone or Dimethylacetamide or combination thereof;
wherein PLGA comprises a molar ratio of 35-85% lactic acid to 65-15% glycolic acid; and
the wt % is on the weight of the liquid injectable composition & wherein the composition is a controlled release composition of unit dosage form.

According to yet another embodiment of the present invention is a method of treating a subject suffering from a disease or disorder ameliorated by donepezil, the method comprising administering the liquid injectable composition of the present invention, in an effective amount and for a period of time sufficient to treat the subject. The subject may be a human.

The disease or disorder may be Alzheimer's, Schizophrenia, Parkinson's Disease or Down Syndrome.

The injectable composition of the present invention may be administered to the subject intramuscularly or subcutaneously.

The administration may be once per 120 days or once per 7 days to once per 120 days or once per 7 days, once per 15 days, once per 21 days, once per 30 days, once per 45 days, once per 90 days or once per 120 days.

In Vitro Release and In Vivo Testing

The development of an in vitro release testing method having a good discrimination capacity is a key part of controlled release formulation development. In vitro release testing is a tool to optimize formulation, as change in the formulation composition or process is reflected in in vitro release testing. For in situ implants, static in vitro testing methods are preferred wherein formulation is added into the media (pH 7.4 buffer with or without surfactants or any other suitable release media) contained in the bottle/vial/falcon tube and maintained at 37° C. using water bath or shaker bath. At predetermined time points aliquot from the media or complete media is removed and replaced with the fresh buffer and analysed using suitable analytical method to detect drug concentration. Other compendial methods of in vitro release testing like rotating basket apparatus, rotating paddle apparatus, reciprocating cylinder, flow through cell apparatus, paddle over disc, reciprocating holder can also be employed.

To predict, accurately and precisely, expected bioavailability characteristics for controlled release formulations from in vitro release profile is difficult and therefore in vivo testing is required to be performed. For in vivo testing, formulations are injected into animals and humans and blood samples are withdrawn at predetermined time points to analyse plasma blood concentration. In vivo plasma concentration profile can provide information regarding bioavailability (AUC), maximum plasma concentration achieved (Cmax), time to reach maximum plasma concentration (Tmax), elimination half-life of drug etc. All these parameters are important to understand safety and efficacy of formulation, Other toxicity evaluation such as organ toxicity, injection site toxicity can also be assessed through in vivo testing.

### Definition of Terms

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “liquid injectable composition” refers to a liquid composition suitable for administration via injection. The injection can be carried out employing a syringe. The route of administration can be parenteral (e.g., intramuscular or subcutaneous). The liquid injectable composition will preferably be sterile.

The term “sterilization” refers to the process that renders specimen free from viable microorganisms

As used herein, the term “gel” refers to a substance having a gelatinous, jelly—like, or colloidal properties.

The term “bioerodible” refers to a substance that when placed in the human body, will break down into individual components.

The term “biodegradable” refers to a substance that when placed in the human body, will break down into components that can readily be eliminated from the body.

The term “release retardant” refers to a substance present in liquid injectable composition that will slow the release of donepezil from the liquid injectable composition when administered, or from the depot formed in situ.

The term “polymer” refers to a substance composed of macromolecules. The polymer can be naturally occurring or synthetic. The polymer can be straight-chained or a branched (e.g., star, comb, etc.) polymer. A polymer which contains only a single type of repeat unit is known as a homopolymer, while a polymer containing two or more types of repeat units is known as a copolymer. A terpolymer is a copolymer which contains three types of repeat units. The polymer can be a homopolymer or copolymer (e.g., statistical copolymer, alternating copolymer, block copolymer, graft copolymer, or gradient copolymer).

The term “macromolecule” refers to a molecule of high relative molecular mass, the structure of which essentially comprises the multiple repetition of units derived, actually or conceptually, from molecules of low relative molecular mass.

The term “polylactic-co-glycolic acid)” or “PLGA” refers to a copolymer composed of lactic acid and glycolic acid monomers. PLGA is typically synthesized by means of ring-opening co-polymerization of two different monomers, the cyclic dimers (1,4-dioxane-2,5-diones) of glycolic acid and lactic acid. During polymerization, successive monomeric units (of glycolic or lactic acid) are linked together in PLGA by ester linkages, thus yielding a linear, aliphatic polyester as a product. The term includes all polymeric forms such as linear, branched, star, random, block, graft, and the like. Depending on the ratio of lactide to glycolide used for the polymerization, different forms of PLGA can be obtained: these are usually identified in regard to the molar ratio of the monomers used (e.g. PLGA 75:25 identifies a copolymer whose composition is 75% lactic acid and 25% glycolic acid). PLGA, as the term is used herein, includes molecular chains with terminal hydroxyl groups, terminal carboxyl groups (i.e., acid-terminated, sometimes termed PLGH) and terminal ester groups (i.e., capped). Terminal ester groups could comprise of ethyl or methyl or hexane diol or other esters. Various polymer properties (degradation and water uptake) are controlled by modifying end-groups: Acid (A) or Ester (E). Poly(lactic-co-glycolic acid) with acid terminal degrades faster as compared to ester terminated one as terminal acid causes faster acid catalyzed hydrolysis of Polylactic-co-glycolic acid) than ester that protects free OH group.

PLGA undergoes hydrolysis in the hod to produce the original monomers: lactic acid and glycolic acid. These two monomers under normal physiological conditions, are by-products of various metabolic pathways in the body. Lactic acid is metabolized in the tricarboxylic acid cycle and eliminated via carbon dioxide and water. Glycolic acid is metabolized in the same way, and also excreted through the kidney. Since the body can metabolize the two monomers, there is minimal systemic toxicity associated with using PLGA for biomaterial applications.

The term. “polylactide” or “PLA” refers to a thermoplastic polyester with backbone formula (C₃H₄O₂)ₙ or [—C(CH₃)HC(═O)O—]ₙ, typically obtained by condensation of lactic acid C(CH₃)(OH)HCOOH with loss of water (hence its name). It can also be prepared by ring-opening polymerization of lactide [—C(CH₃)HC(═O)O—]₂, the cyclic dimer of the basic repeating unit. The name “polylactic acid” does not comply with IUPAC standard nomenclature, and is potentially ambiguous or confusing, because PLA is not a polyacid (polyelectrolyte), but rather a polyester.

The term “number average molecular weight” or “Mn” refers to the sum of all molecular weights divided by their total number of molecules. The term “weight average molecular weight” or “Mw” refers to the sum of all molecular weights multiplied by their weight fractions. The polymerization process, whether proceeding by chain growth or by step-growth, is ruled by random events. The result is a mixture of polymers that vary in chain length. A polymeric material, therefore, cannot be characterized by a single molecular weight like an ordinary substance. Instead, a statistical average calculated from the molecular weight distribution has to be used. The average can be expressed in two ways: number average and weight average.

The term “Polydispersity index” or “PDI” refers to the ratio of weight average molecular weight (Mw) to number average (Mn), PDI=Mw/Mn.

PDI indicates distribution of polymer chain molecular weights in a given polymer. The term “Polydispersity index” (PDI) indicates the breadth of the molecular weight distribution of polymer and therefore could influence reproducibility of drug release from the liquid injectable composition.

The term “inherent Viscosity (Rig)” of polymer is a measure of its molecular weight. (e.g. a polymer with a high inherent viscosity has a higher molecular weight and longer degradation time). The control of molecular weight and/or inherent viscosity of the polymer is a factor involved in the formation and performance of the implant. In general, polymers with higher molecular weight and higher inherent viscosity should provide an implant with a slower degradation rate and therefore a longer duration.

The term “biocompatible” refers to a substance's ability to be in contact with human tissue without producing an adverse effect.

The term “solvent” refers to substance that dissolves a solute, resulting in a solution. As used herein, the solvent will be a liquid and will dissolve substances such as a polymer and donepezil, to provide the liquid injectable composition. The solvent can be polar or non-polar and can be protic or aprotic. The solvent may be selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof. Preferred solvents include polar aprotic solvents such as N-methyl pyrrolidone, N,N-dimethyl acetamide, dimethylformamide, glycerol formal, propylene glycol, benzyl alcohol. Most preferred solvents include N-methyl pyrrolidone and N,N-dimethyl acetamide.

The term “N-methylpyrrolidone” or “N Methyl-2-pyrrolidone” refers to the compound having the IUPAC name 1-methylpyrrolidin-2-one, CAS number 872-50-4, chemical formula C₅H₉NO, and molar mass 99.133 g/mol.

The term “dimethylsulfoxide” refers to the organosulfur compound with the formula. (CH₃)₂SO, CAS number 67-68-5, chemical formula C₂H₆OS, and molar mass 78.13 g/mol.

The term “2-pyrrolidone” refers to the organic compound having the IUPAC name pyrrolidin-2-one, CAS number 616-45-5, chemical formula C₄H₇NO, and molar mass 85.106 g/mol.

The term “N,N-dimethyl acetamide” refers to the organic compound with the formula CH₃C(O)N(CH₃)₂, CAS number 127-19-5, chemical formula C₄H₉NO, and molar mass 87.122 g/mol.

The term “dimethylformamide” refers to the organic compound with the formula (CH₃)₂NC(O)H, CAS number 68-12-2, chemical formula C₃H₇NO, and molar mass 73.095 g/mol.

The term “glycofurol” or “tetraglycol” refers to the organic compound having the name tetrahydrofurfuryl alcohol polyethyleneglycol ether and CAS number 31692-85-0.

The term “glycerol formal” refers to the mixture of 1,3-dioxan-5-ol and 4-hydroxymethyldioxolane; CAS number 99569-11-6, chemical formula C₈H₁₆O₆_and molar mass 208.21 g/mol.

The term “propylene glycol” refers to the compound having the IUPAC name propane-1,2-diol, CAS number 57-55-6, chemical formula C₃H₅O₂, and molar mass 76.095 g/mol.

The term “benzyl benzoate” refers to the compound having the CAS number 120-51-4, formula C₁₄H₁₂O₂, and molar mass 212.248 g/mol.

The term “benzyl alcohol” refers to an aromatic alcohol with the formula C₆H₅CH₂OH. The benzyl group is often abbreviated “Bn” (not to be confused with “Br” which is used for benzoyl), thus benzyl alcohol is denoted as BnOH. Benzyl alcohol has the IUPAC, name phenylmethanol, CAS number 100-51-6, chemical formula C₇H₈O, and molar mass 108.140 g/mol.

The term “donepezil” refers to the compound having the IUPAC name (RS)-2-[(1-benzyl-4-piperidyl)methyl]-5,6-dimethoxy-2,3-dihydroinden-1-one; CAS number 120014-06-4; chemical formula C₂₄H₂₉NO₃; and molar mass 379.500 g/mol. The donepezil can exist in the free base form, or as a pharmaceutically acceptable salt with a suitable counter ion.

The term “stabilizing agent” refers to a substance that inhibits, prevents, decreases, or slows the process of degradation of the liquid injectable composition, or any substance(s) present therein. For example, antioxidants like vitamin A, vitamin E, vitamin C, vitamin C palmitate, retinyl palmitate, and selenium; the amino acids cysteine and methionine; citric acid and sodium citrate.

The term “crystallization inhibitor” refers to a substance that inhibits, prevents, decreases, or slows the process of crystallization of any substance present in the liquid injectable composition. For example, benzyl alcohol, tween 80, PEG, poloxamer etc.

The term “pH adjusting agent” refers to a substance that, when added to an aqueous solution, will change the pH. For example, the pH adjusting agent can be an acid, such that when added to an aqueous solution, it will decrease the pH. Alternatively, the adjusting agent can be a base, such that when added to an aqueous solution, it will increase the pH. The base can be an inorganic base (e.g., sodium hydroxide), and the acid can be at least one of an inorganic acid (e.g., hydrochloric acid) and/or an organic acid (e.g., citric acid, malic acid, tartaric acid, etc.).

The term “subject” is used herein to generally include humans. The subject can be a patient, e.g., for the treatment of Alzheimer's Disease, Schizophrenia, Parkinson's Disease or Down Syndrome.

The term “buffer” refers to a weak acid or base or a mixture of weak acid and its salt or a mixture of a weak base and its salt used to maintain the acidity (pH) of a solution near a chosen value after the addition of another acid or base. That is, the function of a buffering agent is to prevent a rapid change in pH when acids or bases are added to the solution.

The term “preservative” refers to a substance that is added to prevent decomposition by microbial growth. Some typical preservatives used in pharmaceutical formulations include synthetic preservatives like the parabens: methyl paraben and propyl paraben.

The term “burst release control additive” refers to a substance present in liquid injectable composition that will slow the release of donepezil from the depot when formed in situ. For example, co-polymer of poly(lactide-coglycolide) and polyethylene glycol (PLG-PEG), PEG-400 monostearate, polyglyceryldistearate, glyceryl monostearate, benzyl benzoate etc.

The term “depot injection” refers to an injection that delivers donepezil into the body over a sustained period of time. The depot injection includes bioerodible and biodegradable polymer, biocompatible solvent, and optionally excipient(s). The bioerodible and biodegradable polymer serves to manage the donepezil's rate of release into the body. Substances delivered by this method can often last for weeks and months, with certain formulas lasting considerably longer.

The term “depot” refers to the mass structure which could be in the form of a gel or a semisolid or solid implant, formed after the liquid injectable composition is delivered within the body. As the liquid injectable composition contacts bodily tissue and fluids, the depot forms, which subsequently erodes over a desired period of time, releasing the donepezil typically through diffusion and/or dissolution into bodily fluids.

The term “in situ” refers to the location, and surrounding region, within the human body in which the liquid injectable composition is administered and the depot subsequently forms.

The term “aqueous fluid” refers to a liquid fluid that contains water.

The term “bodily fluid” refers to liquids within the human body and include, e.g., blood.

The term “modified release” (in contrast to immediate-release) refers to the delivery of donepezil with a delay after its administration (delayed-release dosage) or for a prolonged period of time (time-release or extended-release [ER, XR, XL] dosage). “Sustained-release” dosage forms refers to dosage forms designed to release (liberate) the donepezil at a predetermined rate in order to maintain a constant drug concentration for a specific period of time with minimum side effects, “Extended-release” dosage consists of either sustained-release (SR) or controlled-release (CR) dosage. SR maintains drug release over a sustained period but not at a constant rate. CR maintains drug release over a sustained period at a nearly constant rate.

The term “burst effect” refers to a relatively large drug release from the depot, over a relatively small period of time. When a burst effect is present, i typically occurs with an initial rapid drug release.

The term “dry powder” refers to a powder that is dry, such that it is a bulk solid composed of many very fine particles that may flow freely when shaken or tilted. Powders are a special sub-class of granular materials, although the terms powder and granular are sometimes used to distinguish separate classes of material. In particular, powders refer to those granular materials that have the finer grain sizes, and that therefore have a greater tendency to form clumps when flowing. Granular material refers to the coarser granular materials that do not tend to form clumps except when wet.

The term “dissolved” refers to a substance that is caused to pass into solution. For example, a solid (e.g., mixture of PLGA, PLA and donepezil) can pass into solution by contacting with a solvent (e.g., N-methyl pyrrolidone). In doing so, the solid is considered to be dissolved in the solvent.

The term “suspension” refers to a heterogeneous mixture that contains solid particles sufficiently large for sedimentation. The particles may be visible to the naked eye, usually must be larger than one micrometer, and will eventually settle, although the mixture is only classified as a suspension when and while the particles have not settled out. A suspension is a heterogeneous mixture in which the solute particles do not dissolve, but get suspended throughout the bulk of the solvent, left floating around freely in the medium. The internal phase (solid) is dispersed throughout the external phase (fluid) through mechanical agitation, with the use of certain excipients or suspending agents. For example, a solvent (e.g., N-methyl pyrrolidone) can contain an undissolved solid (e.g., donepezil hydrochloride), In doing so, the solid is considered to suspended in the solvent.

The term “unit dosage form” refers to a pharmaceutical drug product in the form in which it is marketed for use, with a specific mixture of active ingredients and inactive components (excipients), in a particular configuration (such as an injectable liquid composition) and apportioned into a particular dose. The term “unit dosage form” includes a single use or single administration unit dosage form.

The term “prefilled injectable syringe” refers to an injectable syringe containing a unit dose of the liquid injectable composition. The syringe is typically fitted with a hypodermic needle and a nozzle to direct the flow into and out of the barrel. As such, the prefilled injectable syringe will typically contain the liquid injectable composition within the barrel of the syringe.

The term “effective amount” is used herein to generally include an amount of active ingredient present; effective for treating or preventing a disease, disorder, or condition in a subject, as described herein.

The term “treating” with regard to a subject, refers to improving at least one symptom of the subject's disease, disorder, or condition. Treating includes curing, improving, or at least partially ameliorating the disease, disorder, or condition, or any of the symptoms thereof.

The term “Alzheimer's”, “Alzheimer's disease” or “AD” refers to a chronic neurodegenerative disease that usually starts slowly and gradually worsens over time. It is the cause of 60-70% of cases of dementia. The most common early symptom is difficulty in remembering recent events. As the disease advances, symptoms can include problems with language, disorientation (including easily getting lost), mood swings, loss of motivation, not managing self-care, and behavioral issues. As a person's condition declines, they often withdraw from family and society. Gradually, bodily functions are lost, ultimately leading to death. Although the speed of progression can vary, the typical life expectancy following diagnosis is three to nine years.

The term “Schizophrenia” refers to chronic disease characterized by deterioration of social contact, cognitive deficits, anxiety and depression, resulting in suicide in about 10% of the schizophrenic population (Lewis & Lieberman, 2000). Schizophrenia affects about 1% of the population (Lewis & Lieberman, 2000), and genetic and environmental factors underlie the eventual eruption of the disease (Ross; 2006).

The term “Parkinson's Disease” (PD) refers to disease that currently affects about 10 million people world-wide. PD is a highly specific degeneration of dopamine-containing cells of the substantia nigra of the midbrain. Degeneration of the substantia nigra in Parkinson's disease causes a dopamine deficiency in the striatum.

The term “Down's syndrome” is the most common disorder of chromosomal abnormalities, mainly caused by trisomy 21 (over 90% of standard trisomy) in chromosome 21 excess.

The term “intramuscular” or “1M” refers to the injection of a substance directly into muscle. In medicine, it is one of several methods for parenteral administration of medications. Muscles have larger and more numerous blood vessels than subcutaneous tissue; intramuscular injections usually have faster rates of absorption than subcutaneous or intradermal injections. Possible sites for 1M injection include deltoid, dorsogluteal, rectus femoris, vastus lateralis and ventrogluteal muscles. The selected site is typically cleansed with an antimicrobial and is allowed to dry. It is injected with the dominant hand using a quick, darting motion perpendicular to the patient's body at an angle between 72 and 90 degrees, as a faster injection is less painful. The needle is then stabilized with the non-dominant hand while the dominant hand slides to the plunger to slowly instill the medication, as a rapid instillation causes more discomfort. The Centre for Disease Control and prevention (CDC) does not recommend the outdated practice of aspirating for blood to rule out injecting into a blood vessel. The needle is withdrawn at the same angle inserted. Using the “Z track” or zigzag technique is recommended, where the skin is pulled and held down to one side with the non-dominant hand about an inch and after the needle is withdrawn the displaced skin is allowed to return to its normal position.

This is to ensure that the medication does not leak back along the needle track. Gentle pressure is applied with a gauze, but the site is not massaged to prevent forcing the medication into subcutaneous tissue.

The term “subcutaneous” refers to the administration as a bolus into the subcutis. the layer of skin directly below the dermis and epidermis, collectively referred to as the cutis. Subcutaneous administration may be abbreviated as SC, SQ, sub-cu, sub-Q, SubQ, or subcut. Subcutaneous tissue has few blood vessels and so drugs injected here are for slow, sustained rates of absorption. It is slower than intramuscular injections but still faster than intradermal injections. Subcutaneous injections are typically inserted at 45″ to 90″ angle, depending on amount of subcutaneous tissue present and length of needle—a shorter, ⅜″ needle is usually inserted 90″ and a ⅝″ needle is usually inserted at 450, The medication is administered slowly, about 10 seconds/milliliter. An 18 to 31 gauge thick needle, ⅜″ to 1″ long needle can also be used. The size is determined by the amount of subcutaneous tissue present, which is based on patient build. The ⅜″ and ⅝″ needles are most commonly used. Suitable injection sites include, e.g., (1) the outer area of the upper arm, (2) the abdomen, from the rib margin to the iliac crest and avoiding a 2-inch circle around the navel. This has the fastest rate of absorption among the sites; (3) the front of the thigh, midway to the outer side, 4 inches below the top of the thigh to 4 inches above the knee. This has a slower rate of absorption than the upper arm; (4) the upper back; and (5) the upper area of the buttock, just behind the hip bone. This has the slowest rate of absorption among the sites.

The term “solid content” refers to the total dissolved and/or dispersed solids in composition with respect to total weight of liquid injectable composition.

### Specific Ranges, Values, and Embodiments

The specific embodiments describing the ranges and values provided below are for illustration purposes only, and do not otherwise limit the scope of the disclosed subject matter, as defined by the claims.

In specific embodiments, the liquid injectable composition includes donepezil free base.

In specific embodiments, the liquid injectable composition includes donepezil hydrochloride.

In specific embodiments, the kit includes donepezil in the form of a dry powder.

In specific embodiments, the kit includes donepezil dissolved or suspended in a biocompatible solvent.

In specific embodiments, the kit includes donepezil dissolved or suspended in a biocompatible solvent that includes dimethylsulfoxide, 2 pyrrolidone, N,N dimethyl acetamide, dimethylformamide, glycofurol, glycerol formal, propylene glycol, benzyl benzoate, N-methyl-2-pyrrolidone, benzyl alcohol, or any combination thereof.

In specific embodiments, the kit includes donepezil (free base or salt) having a particle size below 500 microns.

In specific embodiments, the kit includes donepezil (free base or salt) having a particle size below 450 microns.

In specific embodiments, the kit includes donepezil (free base or salt) having a particle size below 400 microns.

In specific embodiments, the kit includes donepezil (free base or salt) having a particle size below 350 microns.

In specific embodiments, the kit includes donepezil (free base or salt) having a particle size below 300 microns.

In specific embodiments, the kit includes donepezil (free base or salt) having a particle size below 250 microns.

In specific embodiments, the kit includes donepezil (free base or salt) having the following particle size distribution (PSD): D₉₀ of not more than 500 microns.

In specific embodiments, the kit includes donepezil (free base or salt) having the following particle size distribution (PSD): D₉₀ of not more than 450 microns.

In specific embodiments, the kit includes donepezil (free base or salt) having the following particle size distribution (PSD): D₉₀ of not more than 400 microns.

In specific embodiments, the kit includes donepezil (free base or salt) having the following particle size distribution (PSD): D₉₀ of not more than 350 microns.

In specific embodiments, the kit includes donepezil (free base or salt) having the following particle size distribution (PSD): D₉₀ of not more than 300 microns.

In specific embodiments, the kit includes donepezil (free base or salt) having the following particle size distribution (PSD): D₉₀ of not more than 250 microns.

In specific embodiments, the kit includes donepezil (free base or salt) having the following particle size distribution (PSD): D₁₀ of not more than 500 microns. D₅₀ of not more than 350 microns, and D₉₀ of not more than 250 microns.

In specific embodiments, the kit includes donepezil (free base or salt) having the following particle size distribution (PSD): D₁₀ of not more than 500 microns, D₅₀ of not more than 300 microns, and D₉₀ of not more than 250 microns.

In specific embodiments, the kit includes crystalline donepezil (free base or salt).

In specific embodiments, the kit includes crystalline donepezil HCl (Form I).

In specific embodiments, the kit includes amorphous donepezil (free base or salt).

In specific embodiments, the kit includes a solvate or hydrate of donepezil (free base or salt).

In specific embodiments, donepezil (free base or salt) powder is sterilized by gamma irradiation, beta irradiation or electronic beam irradiation process.

In specific embodiments, Sterilization of liquid injectable composition comprising of PLGA or PLA or combination thereof and donepezil (free base or salt) in the solvent was accomplished by a process selected from gamma irradiation, electronic, beam irradiation or by aseptic filtration process through a membrane filter of 0.22 micron pore size.

In specific embodiments, sterilization of solution of PLGA or PLA or combination thereof in the solvent was accomplished by a process selected from gamma irradiation, electronic beam irradiation or by aseptic filtration process through a membrane filter of 0.22 micron pore size.

In specific embodiments, Sterilization of solution of donepezil (free base or salt) in the solvent was accomplished by a process selected from gamma irradiation, electronic beam irradiation or by aseptic filtration process through a membrane filter of 0.22 micron pore size.

In specific embodiments, the kit is in a unit dosage form.

In specific embodiments, the kit is in a unit dosage form, which is a single dose.

In specific embodiments, the kit is in a unit dosage form, wherein the first container is a prefilled injectable syringe, the second container is a prefilled injectable syringe, or both the first container and the second container are prefilled injectable syringes.

In specific embodiments, the kit is configured such that the contents of the first container and the contents of the second container can be sufficiently mixed together to form a liquid injectable composition to be administered after the mixing.

In specific embodiments, the kit is configured such that the contents of the first container and the contents of the second container can be sufficiently mixed together to form a liquid injectable composition to be administered within 15 minutes after the mixing.

In specific embodiments, the subject is a human.

In specific embodiments, the liquid injectable composition further includes at least one of stabilizing agent, crystallization inhibitor, pH adjusting agent, buffer, preservative, and burst release control additive.

In specific embodiments, the liquid injectable composition is at least one of (a)-(j): (a) liquid injectable dosage form, capable of forming a depot in situ; (b) liquid injectable dosage form, capable of forming a depot in situ, which releases the donepezil over a period of time; (c) liquid injectable dosage form, capable of forming a gel in situ in contact with aqueous fluids (d) liquid injectable dosage form, capable of forming a gel in situ, in contact with aqueous fluid and releases the donepezil over a period of time; (e) time release liquid injectable dosage form (f) controlled release liquid injectable dosage form; (g) modified release liquid injectable dosage form; (h) sustained release liquid injectable dosage form; (i) extended release liquid injectable dosage form; and (j) liquid injectable dosage form, capable of forming a depot in situ with a reduced burst effect.

In specific embodiments, the liquid injectable composition includes donepezil free base in 3-30% (w/w).

In specific embodiments, the liquid injectable composition includes donepezil free base in 4-25% (w/w).

In specific embodiments, the liquid injectable composition includes donepezil hydrochloride in 3-30% (w/w).

In specific embodiments, the liquid injectable composition includes donepezil hydrochloride in 4-25% (w/w).

In specific embodiments, the liquid injectable composition includes N-methyl pyrrolidone in 25-94% (w/w).

In specific embodiments, the liquid injectable composition includes N-methyl pyrrolidone in 40-90% (w/w).

In specific embodiments, the liquid injectable composition includes PLA in 3-45% (w/w).

In specific embodiments, the liquid injectable composition includes PLA in 4-23% (w/w).

In specific embodiments, the liquid injectable composition includes PLEA in 3-45% (w/w).

In specific embodiments, the liquid injectable composition includes PLGA in 4-34%. (w/w).

In specific embodiments, the liquid injectable composition includes 35:65 to 85:15 PLGA, such that the copolymer has a molar ratio of 35-85% lactic acid and a molar ratio of 15-65 glycolic acid

In specific embodiments, the liquid injectable composition includes PLGA, PLA, or PLGA PLA, present in 3-45 wt % of the liquid injectable composition.

In specific embodiments, the liquid injectable composition includes Poly(lactic-co-glycolic acid) weight average molecular weight in 7 to 151) kilodalton (kDa).

In specific embodiments, the liquid injectable composition includes Poly(lactic-co-glycolic acid) weight average molecular weight in 10 to 80 kDa.

In specific embodiments, the liquid injectable composition includes Poly(lactic-co-glycolic acid) number average molecular weight 2-70 kDa.

In specific embodiments, the liquid injectable composition includes Poly(lactic-co-glycolic acid) number average molecular weight in 3-50 kDa.

In specific embodiments, the liquid injectable composition includes Polylactic-co-glycolic acid) polydispersity index in 0.5 to 5.

In specific embodiments, the liquid injectable composition includes Poly(lactic-co-glycolic acid) polydispersity index in 0.5 to 4.

In specific embodiments, the liquid injectable composition includes Poly(lactic-co-glycolic acid) inherent viscosity (IV) in 0.05 to 8 dL/g.

In specific embodiments, the liquid injectable composition includes total solid contents in 10-75 (w/w).

In specific embodiments, the liquid injectable composition includes total solid contents in 15-65 (w/w).

In specific embodiments, the liquid injectable is administrated no more than once per 7 days.

In specific embodiments, the liquid injectable is administrated at least once per 120 days.

In specific embodiments, the liquid injectable is administrated once per 7 days, once per 15 days, once per 21 days, once per 30 days, once per 45 days, once per 60 days, once per 90 days, or once per 120 days.

In specific embodiments, the liquid injectable is administrated once per 7 days.

In specific embodiments, the liquid injectable is administrated once per 120 days.

In specific embodiments, the liquid injectable has a mass, per dose, of up to 3 grams.

In specific embodiments, the liquid injectable has a mass, per dose, of at least 0.3 grams.

In specific embodiments, the liquid injectable has a mass (g), per dose, of 0.3-3.

In specific embodiments, the liquid injectable has a mass (g), per dose, of 0.35-3.

In specific embodiments, the liquid injectable has a volume, per dose, of up to 3 mL.

In specific embodiments, the liquid injectable has a volume, per dose, of at least 0.3 mL.

In specific embodiments, the liquid injectable has a volume (mL), per dose, of 0.3-3.

In specific embodiments, the liquid injectable has a volume (mL), per dose, of 0.35-3.

In specific embodiments, the liquid injectable has a ratio of donepezil:PLGA of 1:1 to 1:3.5.

### DRAWINGS

The patent or application file contains at least one drawing executed in color. Copies of this patent or patent application publication with color drawing(s) will be provided by the Office upon request and payment of the necessary fee.

FIG. 1 depicts In vitro release profiles of various controlled release donepezil in situ depot forming systems from Example 2.

FIG. 2 depicts In vitro release profiles of various donepezil in situ depot forming systems from Example 3.

FIG. 3 depicts In vitro release profiles of various donepezil n situ depot forming systems from Example 4.

FIG. 4 depicts In vitro release profiles of various donepezil in situ depot forming systems from Example 4.

FIG. 5 depicts in vitro release profiles of various donepezil in situ depot forming systems from Example 4.

FIG. 6 shows Injection site in rats showing presence of donepezil in situ depot up to 42 days (white circle).

The following examples illustrate preferred embodiments in accordance with the present invention without limiting the scope of the invention.

### EXAMPLES

### Example 1: Donepezil Controlled Release In Situ Depot Forming Systems

### Method of Preparation:

PLEA and donepezil base was dissolved in N-methyl pyrrolidone. Solution was filled into vial and sealed and stored at 5° C. Solution was observed for crystallization and observations were noted.

TABLE 1
Different compositions of donepezil controlled release in situ depot forming systems
Composition in % w/w

| Example No | 1.1 | 1.2 | 1.3 | 1.4 | 1.5 | 1.6 | 1.7 | 1.8 | 1.9 | 1.10 | 1.11 | 1.12 |
|---|---|---|---|---|---|---|---|---|---|---|---|---|
| Donepezil base | 22 | 21 | 18 | 21 | 20 | 18 | 15 | 7 | 20 | 15 | 10 | 7 |
| PLGA 50: 50 E | 22 | 21 | 18 | 26 | 27 | 27 | 28 | 34 | 0 | 0 | 0 | 0 |
| IV: 0.16-0.24 | | | | | | | | | | | | |
| Mw: 13-14 kDa | | | | | | | | | | | | |
| PDI: 2.1-2.4 | | | | | | | | | | | | |
| PLGA 50: 50E | 0 | 0 | 0 | 0 | 0 | 0 | 0 | 0 | 27 | 0 | 0 | 0 |
| IV: 0.32-0.44 | | | | | | | | | | | | |
| Mw: 35.8 kDa | | | | | | | | | | | | |
| PDI; 3 | | | | | | | | | | | | |
| PLGA 50: 50 E | 0 | 0 | 0 | 0 | 0 | 0 | 0 | 0 | 0 | 21 | 0 | 0 |
| IV: 0.75-0.88 | | | | | | | | | | | | |
| PLGA 75: 25 E | 0 | 0 | 0 | 0 | 0 | 0 | 0 | 0 | 0 | 0 | 10 | 0 |
| IV: 0.8-1.2 | | | | | | | | | | | | |
| Mw: 127 kDa | | | | | | | | | | | | |
| PLGA 85: 15 E | 0 | 0 | 0 | 0 | 0 | 0 | 0 | 0 | 0 | 0 | 0 | 7 |
| IV: 0 1.3-1.7 | | | | | | | | | | | | |
| N-methyl | 56 | 58 | 64 | 53 | 53 | 55 | 57 | 59 | 53 | 64 | 80 | 86 |
| pyrrolidone | | | | | | | | | | | | |
| Solid content | 44 | 42 | 36 | 47 | 47 | 45 | 43 | 41 | 47 | 36 | 20 | 14 |
| Visual observation | Crystal- | Clear | Clear | Crystal- | Crystal- | Clear | Clear | Clear | Clear | Clear | Clear | Clear |
| | lization | | | lization | lization | | | | | | | |

IV = inherent viscosity in dL/gm
E: Ester end capped

### Example 2: Preparation and Evaluation of In Vitro Release of Controlled Release In Situ Depot Forming Systems of Donepezil

### Method of Preparation:

Method of preparation is same as example 1 except 2.12, 2.13 and 2.14.

Example 2.12, 2.13 and 2.14, PLEA was dissolved in N-methyl pyrrolidone and then donepezil hydrochloride was dispersed. Dispersion was filled into the vial, sealed and stored.

### Method to Study In Vitro Release:

Donepezil controlled release in situ depot forming system was added to release media (USP Phosphate buffer (pH 7.4) (50 mM)+0.25% w/v SLS+0.02% Sodium azide) maintained at 37° C. Aliquot was withdrawn at 8 h, day 1, 3, 7, 15, 21, 30 (for example 2.1, 2.3, 2.4, 2.6, 2.7 and 2.8) and extra time points were withdrawn on day 45 for example 2.9, day 50 and 70 for example 2.10. Aliquots were diluted appropriately, and absorbance was measured using UV spectrophotometer.

TABLE 2
Different compositions of controlled release donepezil in situ depot forming
systems prepared for in vitro release testing:
Compositions in % w/w

| Example No | 2.1 | 2.2 | 2.3 | 2.4 | 2.5 | 2.6 | 2.7 | 2.8 | 2.9 | 2.10 | 2.11 | 2.12 | 2.13 | 2.14 |
|---|---|---|---|---|---|---|---|---|---|---|---|---|---|---|
| Donepezil base | 25 | 10 | 21 | 15 | 10 | 15 | 13 | 15 | 13 | 21 | 12 | 0 | 0 | 0 |
| Donepezil HCl | 0 | 0 | 0 | 0 | 0 | 0 | 0 | 0 | 0 | 0 | 0 | 17 | 25 | 8 |
| PLGA 50: 50 E; | 25 | 0 | 0 | 0 | 27 | 0 | 0 | 0 | 0 | 0 | 34 | 0 | 0 | 0 |
| IV: 0.16-0.24 | | | | | | | | | | | | | | |
| Mw: 13-14 kDa | | | | | | | | | | | | | | |
| PDI: 2.1-2.4 | | | | | | | | | | | | | | |
| PLGA 50: 50 E; | 0 | 0 | 21 | 29 | 0 | 0 | 0 | 0 | 0 | 0 | 0 | 25 | 25 | 23 |
| IV: 0.32-0,44 | | | | | | | | | | | | | | |
| Mw: 35.8 kDa | | | | | | | | | | | | | | |
| PDI; 3 | | | | | | | | | | | | | | |
| PLGA 50: 50 E; | 0 | 27 | 0 | 0 | 0 | 29 | 0 | 0 | 0 | 0 | 0 | 0 | 0 | 0 |
| IV: 0.63-0.77 | | | | | | | | | | | | | | |
| Mw: 42.3 kDa | | | | | | | | | | | | | | |
| PDI: 1.66 | | | | | | | | | | | | | | |
| PLGA 50: 50 E; | 0 | 0 | 0 | 0 | 0 | 0 | 27 | 0 | 0 | 0 | 0 | 0 | 0 | 0 |
| IV: 0.75-0.86 | | | | | | | | | | | | | | |
| Mw: 93.1 k | | | | | | | | | | | | | | |
| PDI: 1.59 | | | | | | | | | | | | | | |
| PLGA 65: 35 E; | 0 | 0 | 0 | 0 | 0 | 0 | 0 | 29 | 0 | 0 | 0 | 0 | 0 | 0 |
| IV: 0. 0.63-0.77 | | | | | | | | | | | | | | |
| Mw: 45.3-48.4 kDa | | | | | | | | | | | | | | |
| PDI: 1.74-1.65 | | | | | | | | | | | | | | |
| PLGA 85: 15 E; | | 0 | 0 | 0 | 0 | 0 | 0 | 0 | 27 | 0 | 0 | 0 | 0 | 0 |
| IV: 0.63-0.77 | | | | | | | | | | | | | | |
| Mw: 80 kDa | | | | | | | | | | | | | | |
| PDI: 1.92 | | | | | | | | | | | | | | |
| PLA E0.3-0.4 | 0 | 0 | 0 | 0 | 0 | 0 | 0 | 0 | 0 | 21 | 0 | 0 | 0 | 0 |
| Mw: 47.2 kDa | | | | | | | | | | | | | | |
| PDI: 1.93 | | | | | | | | | | | | | | |
| N-methyl | 50 | 63 | 58 | 56 | 63 | 56 | 60 | 56 | 60 | 58 | 54 | 58 | 50 | 69 |
| pyrrolidone | | | | | | | | | | | | | | |
| Solid content | 50 | 37 | 42 | 44 | 37 | 44 | 40 | 44 | 40 | 42 | 46 | 42 | 50 | 31 |

IV = inherent viscosity in dL/g
E: Ester end capped

Example 2.1, 2.7 showed 21-day release profile whereas example 2.3, 2.4, 2.6 and 2.8 exhibited 0.30-day release profile. Example 0.9 showed 84% release till day 45 and example 2, 10 showed 67.21% release till day 70.

Composition in % w/w

| Example No | 3.1 | 3.2 | 3.3 | 3.4 | 3.5 | 3.6 | 3.7 | 3.8 | 3.9 |
|---|---|---|---|---|---|---|---|---|---|
| Donepezil Base | 12 | 13 | 11 | 12 | 13 | 13 | 12 | 16 | 12 |
| PLGA 50: 50 E, | 0 | 0 | 0 | 0 | 33 | 0 | 0 | 0 | 0 |
| IV: 0.35-0.41 | | | | | | | | | |
| Mw 21.7 kDa | | | | | | | | | |
| PDI: 1.82 | | | | | | | | | |
| PLGA 50: 50 E; | 25 | 0 | 0 | 0 | 0 | 0 | 0 | 0 | 29 |
| IV: 0.32-0.44 | | | | | | | | | |
| Mw: 35.8 kDa | | | | | | | | | |
| PDI: 3 | | | | | | | | | |
| PLGA 50: 50 E; | 0 | 0 | 0 | 23 | 0 | 0 | 0 | 0 | 0 |
| IV: 0.63-0.77 | | | | | | | | | |
| Mw: 42.3 kDa | | | | | | | | | |
| PDI: 1.66 | | | | | | | | | |
| PLGA 65: 35 E; | 0 | 0 | 0 | 0 | 0 | 27 | 0 | 0 | 0 |
| IV: 0.63-0.77 | | | | | | | | | |
| Mw: 45.3-48.4 kDa | | | | | | | | | |
| PDI: 1.74-1.65 | | | | | | | | | |
| PLGA 75: 25 E: | 0 | 14 | 0 | 0 | 0 | 0 | 25 | 0 | 0 |
| IV: 0.4-0.6 | | | | | | | | | |
| PLGA 85: 15 E; | 0 | 0 | 22 | 0 | 0 | 0 | 0 | 32 | 0 |
| IV: 0.35-0.45 | | | | | | | | | |
| N-methyl Pyrrolidone | 63 | 31 | 50 | 65 | 54 | 60 | 63 | 52 | 59 |
| Benzyl Alcohol | 0 | 42 | 0 | 0 | 0 | 0 | 0 | 0 | 0 |
| Dimethyl Acetamide | 0 | 0 | 17 | 0 | 0 | 0 | 0 | 0 | 0 |
| Solid content | 37 | 27 | 33 | 35 | 46 | 40 | 37 | 48 | 41 |

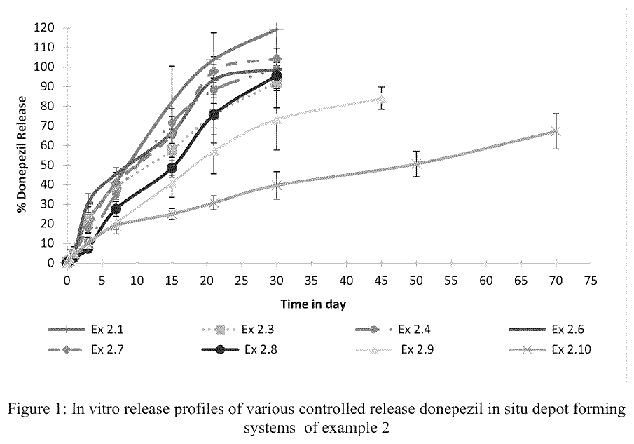

FIG. 1 depicts In vitro release profiles of various controlled release donepezil in situ depot forming systems from Example 2.

### Example 3: Preparation and Evaluation of In Vitro Release of Controlled Release Donepezil In Situ Depot Forming System

### Table 3: Different Compositions of Controlled Release Donepezil In Situ Depot Forming Systems Prepared for In Vitro Release Testing:

IV=inherent viscosity in dL/g
E: Ester end capped
Method of preparation:

in example 3, 3.2 and 3.3; donepezil base was dissolved in solvent. PLGA dissolved in remaining quantity of solvent. Solutions were filled into vial and sealed.

In example 3.4 to 3.9, donepezil powder was sifted through 100 mesh, PLGA was dissolved in N-methyl pyrrolidone then filled into vial and sealed.

Drug solution or drug powder and PLGA solution mixed together prior to in vitro release study.

### In Vitro Release:

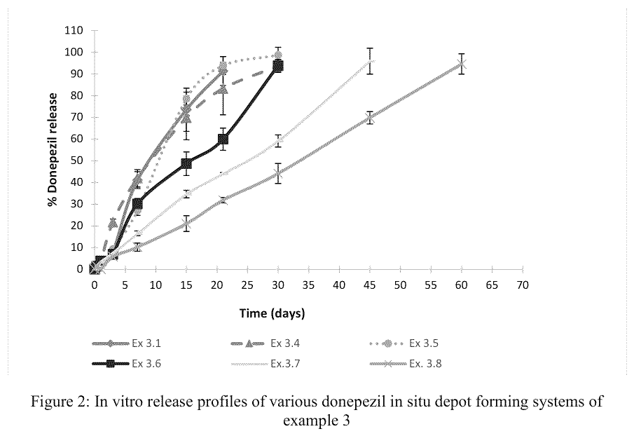

In vitro study was performed as given in Example 2. Example 3.1, 3.4 and 3.5 exhibited complete donepezil base release in 21-30 days. Example 3.6 showed complete donepezil release in 30 days whereas example 3.7 and example 3.8 showed complete donepezil release in 45 and 60 days respectively. FIG. 2 depicts In vitro release profiles of various donepezil in situ depot forming systems from Example 3.

### Example 4: Different Compositions of Controlled Release Donepezil In Situ Depot Forming Systems Prepared for In Vitro Release Testing and In Vivo Study

TABLE 4
Different compositions of controlled release donepezil in situ depot forming systems
prepared for in vitro release testing and for in vivo study
Composition in % w/w

| Example No | 4.1 | 4.2 | 4.3 | 4.4 | 4.5 | 4.6 | 4.7 | 4.8 | 4.9 | 4.10 | 4.11 | 4.12 | 4.13 |
|---|---|---|---|---|---|---|---|---|---|---|---|---|---|
| Donepezil Base | 12 | 11 | 13 | 15 | 14 | 16 | 18 | 14 | 11 | 25 | 20 | 14 | 13 |
| PLGA 65: 35 E; | 31 | 27 | 26 | 30 | 26 | 25 | 25 | 20 | 15 | 25 | 20 | 0 | 0 |
| IV: 0.63-0.77 | | | | | | | | | | | | | |
| Mw: 47.5-48.4 | | | | | | | | | | | | | |
| PDI: 1.71-1.65 | | | | | | | | | | | | | |
| PLGA 75: 25E | 0 | 0 | 0 | 0 | 0 | 0 | 0 | 0 | 0 | 0 | 0 | 28 | 0 |
| IV: 0.25-0.35 | | | | | | | | | | | | | |
| Mw: 23.2 | | | | | | | | | | | | | |
| PDI: 1.97 | | | | | | | | | | | | | |
| PLGA 50: 50E: | 0 | 0 | 0 | 0 | 0 | 0 | 0 | 0 | 0 | 0 | 0 | 0 | 26 |
| IV: 0.32-0.44 | | | | | | | | | | | | | |
| Mw: 36.1 | | | | | | | | | | | | | |
| PDI: 2.9 | | | | | | | | | | | | | |
| N-methyl pyrrolidone | 57 | 62 | 61 | 55 | 60 | 59 | 57 | 66 | 74 | 50 | 60 | 58 | 61 |
| Total | 100 | 100 | 100 | 100 | 100 | 100 | 100 | 100 | 100 | 100 | 100 | 100 | 100 |
| Total solid content | 43 | 38 | 39 | 45 | 40 | 41 | 42 | 34 | 26 | 50 | 40 | 42 | 39 |

IV = inherent viscosity in dL/g
E: Ester end capped

### Method of Preparation:

In example 4.1 to 4.13, donepezil powder was sifted through 100 mesh and filled into vials. Vials were terminally sterilized with gamma irradiation. PLGA was dissolved in N-methyl pyrrolidone and sterilized by filtration through 0.22 micron filter. Then filled into vial and sealed.

Drug powder and PLEA solution mixed together prior to in vitro release and in vivo animal study.

### In Vitro Release:

In vitro study was performed as given in Example 2.

All examples except example no 4.9 showed less than 5% release at day 1, this suggests that control on burst release was achieved. Example 4.2, 4.3, 4.5, 4.6, 4.7, 4.9, 4.10, 4.12 and 4.13 exhibited complete donepezil release (>85%) in 30 days. Example 4.1, 4.4, 4.8 and 4.11 showed around 70-79% release on day 30.

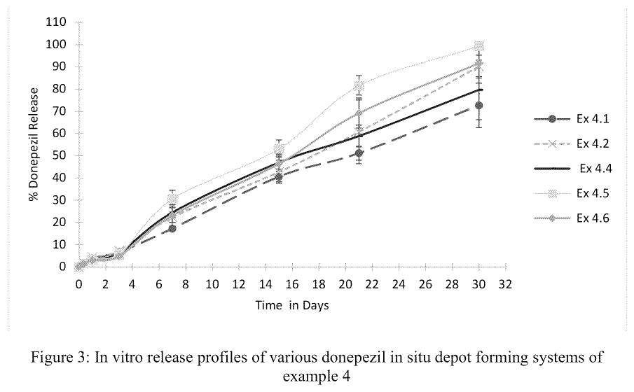

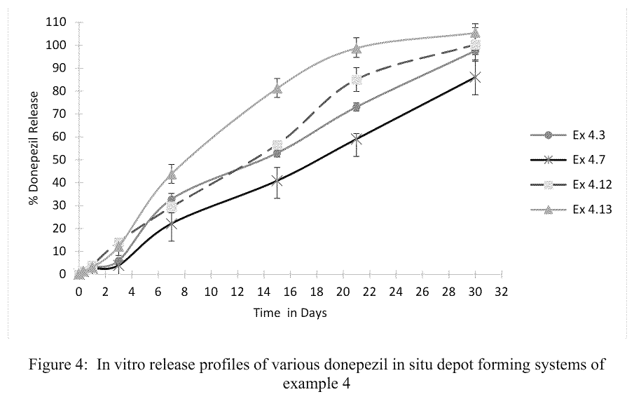

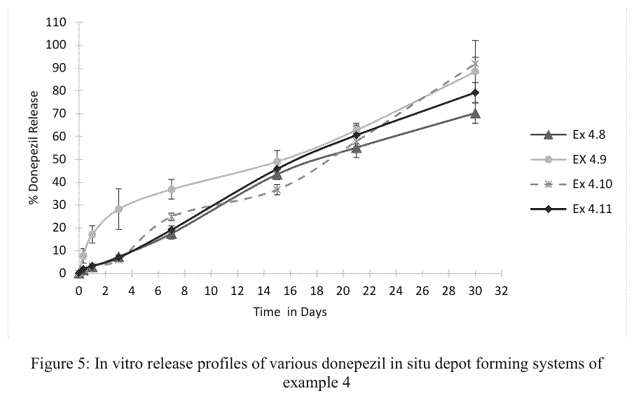

FIGS. 3, 4 and 5 depict in vitro release profiles of various donepezil in situ depot forming systems from Example 4

### Example 5: In Vivo Study of Donepezil In Situ Forming Depot System in Rat

In vivo study was conducted in Sprague Dawley Rats (200-250 g). Six rats per donepezil in situ forming depot of example no. 4.3, 4.7, 4.12 and 4.13 were injected subcutaneously in dorsal area near the neck with 50 mcl of donepezil in situ forming depot described above.

During the course of the study, the animals were observed for activity, weight change, changes at site of injection including redness, bleeding, swelling, discharge, bruising at the injection site were observed and recorded. In addition, injection weights were recorded at administration and body weights were taken and recorded at administration, weekly thereafter and at termination.

At selected time points up to 42 days, six rats per donepezil in situ depot were anesthetized and bled (about 0.2 mL) via retro orbital plexus. The blood was centrifuged for 10 min at 5000 rpm. The plasma fraction was transferred to labelled 1 mL plastic micro centrifuge tubes and stored at −86° C. The plasma was extracted, and donepezil plasma concentration data is being generated using LC-MS/MS technique.

At 42 days the animals were euthanized, and injection site tissues were removed. Injection site tissues were observed visually for presence of implant and photographic images were taken. The injection site tissue (3 samples/test article) was sent for histopathology study. Inflammatory reaction, leucocytic infiltration, fibroplasia, laid down collagen, neovascularization and presence of implant at injection site was checked and recorded during histopathology study.

### Observation:

The study observations are as below:

TABLE 3
Visual and histopathological observations from in vivo rat study:

| | | observation of | | | | | |
|---|---|---|---|---|---|---|---|
| | Weight | implant at | | | | | |
| Ex. | gain and | injection site on | Leucocytic | | Laid down | | Grade of |
| no | behavior | day 42 | Infiltration | Fibroplasia | Collagen | Neovascularization | reaction |
| 4.3 | Normal | Implants still | Moderate to | Mild to | Not seen | Mild to moderate | Moderate to |
| | | visible in 5 out | severe, large | moderate | | (2 out of 3) | marked |
| | | of 6 rats | lymphocytic | (2 out of 3) | | | (2 out of 3) |
| | | | and few | | | | |
| | | | macrophages | | | | |
| | | | (1 out of 3) | | | | |
| 4.7 | Normal | Implant still | NAD | NAD | NAD | NAD | NAD |
| | | visible in 2 out | | | | | |
| | | of 6 rats | | | | | |
| 4.12 | Normal | Implant still | NAD | NAD | NAD | NAD | NAD |
| | | visible in 4 out | | | | | |
| | | of 5 rats | | | | | |
| 4.13 | Normal | No implant | NAD | NAD | NAD | NAD | NAD |
| | | visible in any | | | | | |
| | | of the 6 rats | | | | | |

NAD: No abnormalities detected

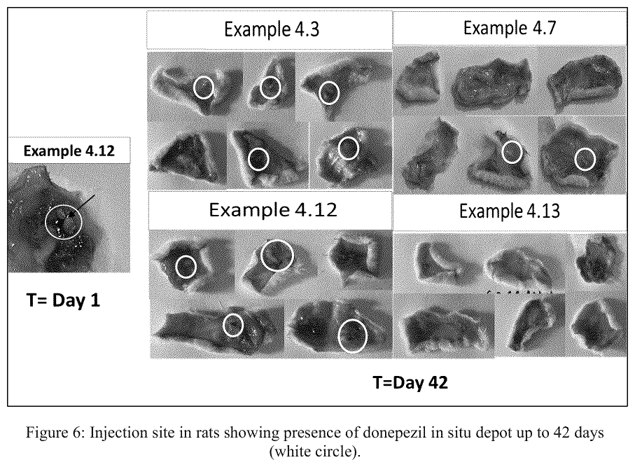

One animal of example 4.12 was euthanized at 24 hours and a formed implant was clearly, seen under the skin at injection site (FIG. 6) We conclude that implant formation has taken place under the skin in rats as seen in example no 4.12.

In example 4.13 no implants were observed after 42 days in all 6 animals implying that the depot implant had bio eroded in this lime frame, Whereas example no, 4.12 (4 out of 5) and 4.3 (5 out of 6) showed presence of implant on day 42.

Example no 4.13 comprises of PLGA 50:50 E which could degrade faster whereas example no 4.3 and example no 4.12 comprise of PLGA having higher lactide content i.e. PLGA 65:35E and PLGA 75:25E respectively.

In example no 4.7, only 2 out of 6 animals showed presence of implant on day 42 this can be attributed to different ratio of Donepezil: PLGA present as compared to example no 4.3.

All rats showed normal behavior, normal weight gain and no redness, bleeding, swelling, discharge, bruising or any other adverse effects at the injection site during the course of study.

The histopathology examination showed no inflammation at injection site for example no 4.7, 4.12 and 4.13 suggesting safety of test articles. In example 4.3 mild inflammation was observed in few rats.

FIG. 6 shows injection site in rats showing presence of donepezil in situ depot up to 42 days (white circle).

1. A liquid injectable composition comprising:
(a) bioerodible and biodegradable release retardant polymer selected from poly(lactic-co-glycolic acid) (PLGA), polylactide or combination thereof;
(b) biocompatible solvent selected from dimethylsulfoxide, pyrrolidone, N,N-dimethyl acetamide, dimethylformamide, glycofurol, glycerol formal, propylene glycol, benzyl benzoate, N-methyl-2-pyrrolidone, benzyl alcohol or combination thereof; and
(c) donepezil, in the free base or pharmaceutically acceptable salts thereof.

2. A liquid injectable composition as claimed in claim 1, having a volume of up to 3.0 ml.

3. A liquid injectable composition as claimed in claim 1, having a mass up to 3 g.

4. A liquid injectable composition as claimed in claim 1, wherein the PLGA comprises a molar ratio of 35-85% lactic acid to 65-15% glycolic acid.

5. A liquid injectable composition as claimed in claim 1, wherein the PLGA, PLA or PLGA PLA is present in about 3-45 wt. % of the liquid injectable composition.

6. A liquid injectable composition as claimed in claim 1, wherein the donepezil is present as the free base.

7. A liquid injectable composition as claimed in claim 1, wherein the donepezil is present as the hydrochloride salt.

8. A liquid injectable composition as claimed in claim 1, wherein the donepezil is present as the free base and the donepezil free base is present in 3-30 wt. % of the liquid injectable composition.

9. A liquid injectable composition as claimed in claim 1, wherein the biocompatible solvent is present in about 25-94 wt. % of the liquid injectable composition.

10. A liquid injectable composition as claimed in claim 1; further comprising at least one of stabilizing agent, crystallization inhibitor; pH adjusting agent, buffer, preservative, and burst release control additive.

11. A liquid injectable composition as claimed in claim 1, which is at least one of (a)-(j):
(a) liquid injectable dosage form, capable of forming a depot in situ;
(b) liquid injectable dosage form, capable of forming a depot in situ, which releases the donepezil over a period of time;
(c) liquid injectable dosage form, capable of forming a gel in situ in contact with aqueous fluids
(d) liquid injectable dosage form, capable of forming a gel in situ, in contact with aqueous fluid and releases the donepezil over a period of time;
(e) time release liquid injectable dosage form;
(f) controlled release liquid injectable dosage form;
(g) modified release liquid injectable dosage form;
(h) sustained release liquid injectable dosage form;
(i) extended release liquid injectable dosage form; and
(j) liquid injectable dosage form, capable of forming a depot in situ with a reduced burst effect.

12. A kit comprising:
(a) a first container comprising donepezil, in the free base or a pharmaceutically acceptable salt thereof optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof, and
(b) a second container comprising a bioerodible and biodegradable, release retardant polymer comprising polylactic-co-glycolic acid) (PLGA), polylactide (PLA), or a combination thereof optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone; benzyl alcohol, or any combination thereof.

13. A kit as claimed in claim 12, wherein the donepezil in the free base or a pharmaceutically acceptable salt thereof is in the form of a dry powder.

14. A kit as claimed in claim 12, wherein the donepezil is dissolved or suspended in a biocompatible solvent comprising dimethylsulfoxide; 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof.

15. A kit as claimed in claim 12, present in a unit dosage form.

16. A kit as claimed in claim 12, present in a unit dosage form, wherein
(a) the first container is a prefilled injectable syringe or ampoule or vial; and
(b) the second container is a prefilled injectable syringe or ampoule or vial.

17. A kit as claimed in claim 12, configured such that the contents of the first container and the contents of the second container can be sufficiently mixed together to form a liquid injectable composition to be administered within 15 minutes after the mixing.

18. A kit as claimed in claim 12, present in a unit dosage form which is a single dose.

19. A liquid injectable composition comprising:
(a) 3-45 wt. % of bioerodible and biodegradable release retardant polymer selected from polylactic-co-glycolic acid) (PLGA), polylactide (PLA) or combination thereof;
(b) 25-94 wt. % of biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol or combination thereof;
(c) 3-30 wt. % of donepezil, in the free base or pharmaceutically acceptable salts thereof; and
wherein PLGA comprises a molar ratio of 35-85% lactic acid to 65-15% glycolic acid; and
the wt % is on the weight of the liquid injectable composition & wherein the composition is a controlled release composition.

20. A kit comprising:
(a) a first container comprising donepezil, in the free base or a pharmaceutically acceptable salt thereof optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof;
(b) a second container comprising a bioerodible and biodegradable, release retardant polymer comprising poly(lactic-co-glycolic acid) (PLGA), polylactide (PLA), or a combination thereof optionally in a biocompatible solvent selected from dimethylsulfoxide, 2-pyrrolidone, N,N-dimethyl acetamide, dimethylformamide, glycofurol, glycerol formal, propylene glycol, benzyl benzoate, N-methyl-2-pyrrolidone, benzyl alcohol, or any combination thereof and wherein PLGA comprises a molar ratio of 35-85% lactic acid to 65-15% glycolic acid; and wherein the composition is a controlled release composition of unit dosage form.

21. A liquid injectable composition comprising:
(a) 3-45 wt. % of polylactic-co-glycolic acid);
(b) 25-94 wt. % of N-methyl-2-pyrrolidone or Dimethylacetamide or combination thereof;
(c) 3-30 wt. % of donepezil, in the free base or pharmaceutically acceptable salts thereof; and
wherein PLGA comprises a molar ratio of 35-85% lactic acid to 65-15% glycolic acid; and the wt % is on the weight of the liquid injectable composition, & wherein the composition is a controlled release composition.

22. A kit comprising:
(a) a first container comprising 3-30 wt. % of donepezil, in the free base or a pharmaceutically acceptable salt thereof optionally in 25-94 wt. % of N-methyl-2-pyrrolidone or Dimethylacetamide or combination thereof;
(b) a second container comprising 3-45 wt. % polylactic-co-glycolic acid optionally in N-methyl-2-pyrrolidone;
wherein PLGA comprises a molar ratio of 35-85% lactic acid to 65-15% glycolic acid; and
the wt % is on the weight of the liquid injectable composition & wherein the composition is a controlled release composition of unit dosage form.

23. A method of treating a subject suffering from a disease or disorder ameliorated by donepezil, the method comprising administering the liquid injectable composition of claim 1, in an amount and for a period of time sufficient to treat the subject.

24. A method of treating a subject as claimed in claim 23, wherein the subject is a human.

25. A method of treating a subject as claimed in claim 23, wherein the disease or disorder is Alzheimer's, Schizophrenia, Parkinson's Disease or Down Syndrome.

26. A method of treating a subject as claimed in claim 23, wherein the administration is intramuscular or subcutaneous.

27. A method of treating a subject as claimed in claim 23, wherein the administration is once per 120 days.

28. A method of treating a subject as claimed in claim 23, wherein the administration is from once per 7 days to once per 120 days.

29. A method of treating a subject as claimed in claim 23, wherein the administration is:
once per 7 days,
once per 15 days,
once per 21 days,
once per 30 days,
once per 45 days,
once per 60 days,
once per 90 days, or
once per 120 days.
